(19) United States
(12) Patent Application Publication
(10) Pub. No.: US 2005/0191322 A1
(43) Pub. Date: Sep. 1, 2005

(54) Compositions for preventing posterior capsular opacification and the use thereof

(76) Inventor: Sverker Norrby, Leek (NL)

Correspondence Address: DINSMORE & SHOHL, LLP, 1900 CHEMED CENTER, 255 EAST FIFTH STREET, CINCINNATI, OH, 45202, US

(21) Appl. No.: 10/677,932

(22) Filed: Oct. 2, 2003

Related U.S. Application Data
(60) Provisional application No. 60/417,092, filed on Oct. 7, 2002.

(30) Foreign Application Priority Data
Oct. 3, 2002 (SE) 0202924-7

(51) Int. Cl. A61K 48/00; A61K 38/16; A61K 31/704; A61K 31/7072; A61K 35/78

(52) U.S. Cl. 424/400; 514/44; 514/34; 514/50; 514/283; 514/410; 424/731; 514/2; 514/629

(57) ABSTRACT

The present invention relates to a composition comprising a sterile ophthalmic composition, which comprises one or more agents capable of inhibiting lens epithelial proliferation dissolved in a physiologically isotonic solution. The present invention also relates to a method of preventing capsular opacification comprising by using said composition. The method comprises the steps of: creating an incision within an eye and a capsulorhexis in a capsular bag of said eye; inserting at least a portion of cannula in said capsulorhexis; separating the natural crystalline lens from the capsular bag by injecting through a cannula a sterile ophthalmic composition in a manner that generates a space between the capsular bag and the natural crystalline lens wherein said composition comprises one or more agents capable of inhibiting lens epithelial proliferation dissolved in a physiologically isotonic solution; retaining the composition in said space between the capsule and the natural crystalline lens for a sufficient time for said composition to effectively kill or render non-proliferative lens epithelial cells in or adjacent to said space; removing the lens and said composition; inserting an implant into the capsular bag.

### FIELD OF THE INVENTION

The present invention relates to a method of introducing one or more agents into an eye during a process of freeing the lens from the capsular bag in order to treat the capsular bag or the cells therein for the prevention of proliferation and/or migration of residual lens epithelial cell and for the prevention of the production of the extracellular matrix by said cells, which all might lead to capsular opacification (CO) after extraction of a non-cataractous or cataractous lens, whether or not an artificial lens has been implanted to replace the natural lens. Furthermore, the invention also relates to a composition used in said process comprising said one or more agents.

### BACKGROUND OF THE INVENTION

Cataract extraction is among the most commonly performed operations in the world. The natural lens is located within a capsular bag, also called lens capsule or capsular sac, which is located in the posterior chamber of the anterior segments of the eye. In order to gain access to the natural lens, an incision is made either in either the clear cornea, at the limbus, or in the sclera of the eye whereby it becomes possible to introduce surgical instruments into the anterior segments of the eye. In the case of cataract extraction, an opening is made in the capsular bag, currently mostly by a capsular a capsulorhexis technique, whereby a portion of the anterior membrane of the capsular bag is torn out to allow insertion of surgical instruments into the capsular bag for the purpose of extraction of the natural lens. The natural lens can be removed through by application of many known techniques, including what is known as phacoemulsification. Phacoemulsification is a method that entails the application of ultrasonic energy or other forms of energy to the natural lens thus breaking said lens into fragments, which can then be aspirated from the capsular bag. The capsular bag remains substantially intact throughout the process of cataract extraction, with the exception of the portion removed to prepare access for the surgical instruments used in the extraction of the natural lens. After the removal of the natural lens (aphakia), an artificial intraocular lens (IOL) implant is implanted within the capsular bag in order to mimic the transparency and the refractive function of a natural lens. Alternatively a lens material is injected to fill the capsular bag and thus an artificial lens is created in situ. Such lenses (ACL) can in addition restore the accommodative function of the natural lens before the onset of presbyopia (loss of ability to accommodate).

In modern cataract extraction surgery, especially with phacoemulsification, one feature of the surgical technique is to separate the natural lens from the capsular bag, one such a technique is hydrodissection. In this technique a fluid wave is injected under the anterior capsule in such a way that it separates the lens from the capsular bag. One of the most common used fluids for the purpose of hydrodissection is a balanced salt solution, which is both ionically and osmotically balanced with regard to the aqueous homour and internal tissue of the eye. In addition to sodium chloride, said solution contains also potassium chloride, calcium chloride, magnesium chloride, sodium acetate and sodium citrate. The balanced salt solutions are considered to be physiologically compatible with the ocular tissue since they contain the essential ions for normal cell metabolism.

Lens removal with IOL or ACL implantation replacement provides significant benefits to most cataract patients. Currently lens removal with artificial lens implantation is increasingly carried out in a non-catarcatous eye, so-called refractive lens exchange, often with the purpose to relieve presbyopia. However, it is estimated that up to fifty percent of all patients, who have implants placed within the capsular bag, will develop capsular opacification (CO), also known as secondary cataract or aftercataract, within five years after surgery. CO is an opacification located on the inner surface of capsular bag, whether located posteriorly (PCO) or anteriorly (ACO). CO is caused by deposition or ingrowth of cells, cell derivatives and/or fibers into the visual axis and might also be caused by extracellular matrix produced by the lens epithelial cells, thereby occluding the optical axis of the eye and thus clouding of the vision. Thus, the cell deposits on the capsule and/or on the implant originate from the proliferation and migration of residual lens epithelial cells on the interior surface of the capsular bag and the production of extracellular matrix by these cells. During cataract surgery, the surgeon removes the lens and replaces it with a new artificial lens.

Ophthalmic surgeons, aware of the problems associated with residual lens epithelial cells, typically take considerable care in trying to remove as many as possible of the lens epithelial cells prior to implantation of an artificial lens (IOL or ACL). However, despite these efforts, a significant number of lens epithelial cells are usually left on the interior surface of the capsular bag since these cells are difficult to view and often difficult to reach and virtually impossible to completely remove.

The most common treatment for postoperative PCO uses laser energy, which is applied to the posterior membrane of the capsular bag for the purpose of creating an opening in the posterior capsule (known as Nd-YAG capsulotomy). However, the laser energy applied to the posterior membrane of the capsular bag is ordinarily directed through the implant and might damage the optic of said implant. Accordingly, it is preferred to prevent the occurrence of CO rather than treating CO at a later date through the application of laser energy. This is especially desirable when the implant is accommodating response to ciliary muscle contraction, in which case a capsulotomy may compromise the accommodative ability of the lens.

Various procedures for the prevention of CO have been suggested in recent years. Many such procedures have included the application of chemicals into of the capsular bag in order to destroy residual lens epithelial cells. However, few if any of these procedures have proven to be particularly successful in the prevention of CO due to the fact that it is extremely difficult to destroy residual lens epithelial cells without simultaneously destroying other cells within the eye, e.g. there exists a number of chemical agents that could kill the lens epithelial cells, however, said agents may also adversely affect other cells with in the eye, in particular corneal endothelial cells. Thus, selective destruction of residual lens epithelial cells by exploitation of the cells increased proliferate activity has thus been the primary approach for the prevention of CO.

Antimetabolites such as 5-fluorouracil (5FU) and daunomycin have been injected into the capsular bags of eyes in attempts to prevent CO. However, for antimetabolite therapy to be effective, the agents must be present when the residual lens epithelial cell proliferation resumes at an indeterminate time following surgery. Sustained drug delivery systems have also been investigated as means for preventing CO. However, the effective time frame within when to apply these agents may likewise be difficult to determine. Thus, timing is difficult in the prevention of CO since it, as mentioned above, is believed to result primarily from the propagation of residual lens epithelial cells of the germinal layer and it is difficult to accurately predict when said cells might start to proliferate and migrate across the capsular bag into the optical zone.

Immunotoxins, which are hybrid molecules composed of monoclonal antibodies chemically linked to toxic moieties, have also been used in the selective destruction of residual lens epithelial cells. The monoclonal antibody directs the toxic moiety to the target cell. The cell then internalizes the immunotoxin, thereby causing vital biological processes of the cell to be compromised by the toxic moiety. Fibroblasti growth factor bonded to a toxic moiety has also been used in order to try to prevent CO. However, both monoclonal antibodies and fibroblast growth factors are relatively expensive and difficult to produce on a reliable and consistent basis.

Patent application WO 02/15828 (Bausch and Lomb) discloses methods for removing epithelial cells by injecting a composition comprising an agent after the natural lens has been removed from the capsular bag. The disadvantage with this technique is that the capsular bag is empty i.e. the whole capsular bag is thus filled with the composition. Thus, much agent is needed and in case of leakage there is a great risk that many cells outside the capsular bag, in particular corneal endothelial cells, may be damaged. Furthermore, the agent is not concentrated to the region of the inner wall of the capsular bag where the CO can be expected to be most severe. Thus, there exists a need for a relatively simple, reliable and effective method of preventing capsular opacification or in patients implanted with artificial lenses following lens extraction.

### BRIEF SUMMERY OF THE INVENTION

The present invention provides a composition and a method for treating capsular opacification that provides a more effective and accurate treatment, while minimizing the risks of affecting other cells within the eye than the lens epithelial cells within the capsular bag.

Accordingly, it is one object of the present invention to provide a sterile ophthalmic composition comprising one or more agents capable of inhibiting lens epithelial proliferation dissolved in a physiologically isotonic solution, preferably said physiological isotonic solution is a balanced salt solution, such as BSS® or BSS Plus® or other similar solutions, however BSS® or BSS Plus® are most preferred.

In one aspect said agent or agents in the composition comprises one or more cytotoxic agents, which are selected from the group comprising of saporin, ricin, methotrexate, 5-fluorouracil, daunomycin, doxorubicin, mitoxanthrone, vinca alkaloids, vinblastine, colchicine, cytochasins, monensin, mitomycin and ouabain, most preferably 5-fluorouracil, saporin, mitomycin or doxorubicin.

In another aspect the claimed composition includes one or more agents comprising a molecule of nucleic acid comprising a gene coding for an agent inducing the death of the lens epithelial cells, under a transcriptional control specific to said cells, which is chosen from among the genes coding for a protein inducing cell death by necrosis and the genes coding for proteins toxic for the lens epithelial cells, preferably said agent is a chosen from a gene coding for an agent inducing apoptosis, or a gene involved in the process of apoptosis, most preferably said agent is chosen from among the genes coding for p53, BAX, FLICE (also called caspase 8) TRAIL and TRAIL-R. Furthermore, said one or more agents include a molecule of nucleic acid comprising a gene coding for an agent inducing the death of the lens epithelial cells, under a transcriptional control specific to said cells.

In still another aspect the claimed composition includes one or more agents comprising a vector comprising a molecule of a nucleic acid selected from any of those mentioned in the paragraph above, said vector is preferably of the adenovirus type.

A further aspect of the claimed composition relates to that said one or more agents comprise one or more basement membrane binding agents conjugated to one or more cytotoxic agents, preferably said basement membrane binding agents is selected from the group consisting of ribosomal inhibitory proteins, antimitotic drugs and ionophores, more preferably from the group consisting of poly-L-lysine, poly-D-lysine, fibronectin, laminin, type I, II, III or IV collagen, thrombospondin, vitronectin, polyarginine and platelet factor IV, conjugated to one or more cytotoxic agents, even more preferably poly-L-lysine or poly-D-lysine. The cytotoxic agents are selected from the group consisting of ribosomal inhibitory proteins, antimitotic drugs and ionophores, most preferably ribosomal inhibitory proteins.

Another aspect of the claimed composition relates to that said one or more agents are surfactants.

Another aspect of the claimed composition relates to that is that said one or more agents are divalent cation chelators.

Another aspect of the claimed composition relates to that said one or more agents are analogs or antibodies directed against cell attachment receptors.

In another object of the present invention relates to a method of preventing capsular opacification that comprises:
a. creating an incision within an eye and a capsulorhexis in a capsular bag of said eye;
b. inserting at least a portion of cannula in said capsulorhexis;
c. separating the natural crystalline lens from the capsular bag by injecting through a cannula a sterile ophthalmic composition in a manner that generates a space between the capsular bag and the natural crystalline lens wherein said composition comprises one or more agents capable of inhibiting lens epithelial proliferation dissolved in a physiologically isotonic solution;
d. retaining the composition in said space between the capsule and the natural crystalline lens, which was formed from (c), for a sufficient time for said composition to effectively kill or render non-proliferative lens epithelial cells in or adjacent to said space;
e. removing the lens and said composition;
f. inserting an implant into the capsular bag.

One aspect of the method is that the composition is usually left in the capsular bag for less than 10 minutes, more preferably less than 3 minutes and most preferably less than 30 seconds.

Another aspect of said method is that the composition of the present method is selected form the compositions disclosed earlier.

Still another aspect of said method is that said implant is a lens, which is a foldable lens or a non-foldable lens made from polymethylmethacrylate, homo or co-polymers of acrylates or methacrylates or other substituted acrylates, whether hydrophobic or hydrophilic or polysiloxane polymers or that said lens comprises injectable ophthalmically material that can undergo crosslinking to a final lens implant following its injection into the capsular bag.

A further aspect of the claimed method is that the method used for separating the lens from the capsular bag most preferably is hydrodissection.

These objects and aspects and other objects aspects and advantages of the present invention, some of which are specifically described and others that are not, will become apparent from the detailed description, example and claims that follow.

### DETAILED DESCRIPTION OF THE INVENTION

The composition and the method of the present invention is intended to be used in an eye during a lens removal process, preferably during hydrodissection, to non-specifically or specifically destroy residual lens epithelial cells on the interior surface of the capsular bag. The method may also be used in an eye to lyse cell walls and/or disrupt cell attachment to the capsular bag. By destroying residual lens epithelial cells disposed on the interior surface of the capsular bag by whatever means, the cells are prevented from proliferating and/or migrating along or across the surface of the capsular bag and/or produce extracellular matrix, which will prevent the formation of capsular opacification.

The sterile ophthalmic composition of the present invention comprises one or more agents capable of inhibiting lens epithelial proliferation dissolved in a physiologically isotonic solution. As mentioned earlier the physiologically isotonic solution is a balanced salt solution, which comprises sodium chloride, said solution contains also potassium chloride, calcium chloride, magnesium chloride, sodium acetate and sodium citrate. The most preferred balanced salt solutions used in the present invention are Alcon BBS® or BSS Plus® (that comprises an additional bicarbonate buffering system and dextrose). However, other known balanced salt solutions such as, but not limited to, Tyrode's solution, Hank's solution or Earle's solution could also be used in the present invention. Furthermore, other sterile physiological isotonic that can be used in the eye solutions and are compatible with ocular tissue can also be used in the present invention. The composition of the present invention comprises, as earlier mentioned, also one or more agents. The one or more agents are selected from cytotoxic agents, a molecule of nucleic acid comprising a gene coding for an agent inducing the death of the epithelial cells, basement membrane binding agents conjugated to cytotoxic agents, surfactants, hypertonic solutions and chemical and enzymatic agents that release lens epithelial cells from the capsular bag membrane. Combinations of these agents would also be conceivable in the present invention.

The cytotoxic agents are selected from the group consisting of saporin, ricin, methotrexate, 5-fluorouracil, daunomycin, doxorubicin, mitoxanthrone, vinca alkaloids, vinblastine, colchicine, cytochasins, monensin, mitomycin and ouabain. These toxins are internalized by lens epithelial cells and inhibit vital cellular processes.

The molecule of the nucleic acid comprises a gene coding for an agent, which will induce death of the epithelial cells, under a transcriptional control specific to these cells. The gene coding is chosen from among those genes coding for a protein inducing cell death by necrosis and from those genes coding for toxic proteins. Preferably said gene is a gene coding for an agent inducing apoptosis, or a gene involved in the process of apoptosis. Even more preferably said gene coding for an agent inducing the death of the crystalline lens cells is chosen from among the genes coding for p53, BAX, FLICE (also called caspase 8), TRAIL and TRAIL-R. Said one or more agents can also be selected from a molecule of nucleic acid comprising a gene coding for an agent inducing the death of the lens epithelial cells, under transcriptional control specific to said cells or from a vector comprising a molecule of any of the nucleic acid disclosed above, the vector being preferably of the adenovirus type. The molecule of nucleic acid is characterized in that the transcriptional control specific to the lens epithelial cells is effected by the promoter of αA crystallin, the promoter of γD crystallin, the promoter of MIP (MP26), or the promoter of a biochemical element specific to the lens epithelial cells and quite particularly the promoter of αA crystallin or the promoter of γD crystallin which are very specific to said cells. One condition of implementation is that the molecule of nucleic acid and in particular of DNA, is carried by a vector. The vector can be for example a synthetic vector, which will transport the molecule of nucleic acid according to the invention either in the form of DNA or in the form of RNA, or a viral vector. As viral vector, a vector can be used that is derived either from a virus of the family of retroviruses of the oncovirinae type (Moloney strain) advantageously used in concentrated viral suspension, or from a virus of the lentiviridae type. The viral vector can also be derived from the associated adenovirus (AAV) or from the virus of the family of the adenoviruses. The whole of a viral vector can be used or just a fragment of the latter insofar as it allows the gene coding for a protein inducing cell death to penetrate into the lens epithelial cells to be destroyed. The vector used is preferably an episomal vector, which thus does not integrate itself in the genome of its target cells. Vectors, which are subjects of the present invention, can for example be prepared as follows: A plasmidic construct of nucleic acid, preferably DNA, is realized, containing a gene coding for an agent inducing the death of the said cells under a transcriptional control specific to said cells in order to obtain the expected molecule of nucleic acid which is isolated. Under preferred conditions of implementation of the process described above, a plasmidic construct of DNA is realized containing a gene coding for a protein inducing apoptosis (such as p53), under a transcriptional control specific to the lens epithelial cells (for example using a promoter specific to said cells, in particular the promoter of αA crystallin or the promoter of γDcrystallin), the gene coding for the protein inducing apoptosis preferably being followed by a polyadenylation sequence. The molecule of DNA described above can then be inserted in a vector such as a vector derived from an adenovirus to obtain the expected vector, which is isolated.

Basement membrane binding agents conjugated to cytotoxic agents are likewise suitable. The conjugated basement membrane binding agent bond with basement membrane within the lens capsule, since the residual lens epithelial cells are disposed on the basement membranes within the lens capsule, the basement membrane binding agent will be in direct contact with the lens epithelial cells when said binding agents are bonded the basement membranes. The cytotoxic agent conjugated with the basement membrane binding agents are thereby present to destroy any migration or proliferating lens epithelial cells. In accordance with the present invention one or more, but preferably one for purposes of simplicity, suitable basement membrane binding agent is conjugated with one or more, but preferably one for purposes of simplicity, cytotoxic agents. The cytotoxic agents are preferably selected from the group consisting of ribosomal inhibitory proteins, antimitotic drugs and ionophores and the one or more basement membrane binding agents are preferably selected from the group consisting of poly-L-lysine, poly-D-lysine, fibronectin, laminin, type I, II, III or IV collagen, thrombospondin, vitronectin, polyarginine and platelet factor IV, conjugated to one or more cytotoxic agents, more preferably are said cytotoxic agents selected from the group consisting of ribosomal inhibitory proteins, antimitotic drugs and ionophores, most preferably ribosomal inhibitory proteins and the one or more basement membrane binding agents are more preferably selected from poly-L-lysine or poly-D-lysine. Ribosomal inhibitory proteins are preferable in the present invention due to the fact that such proteins contain more inhibitory activity per microgram than other cytotoxic agents that can be used in connection with the method of the present invention. However, other suitable cytotoxic agents are e.g. antimitotic drugs such as methotrexate, 5-fluorouracil, daunomycin, doxorubicin, mitoxanthrone, vinca alkaloids, vinblastine, colchicine, and cytochasins, and ionophores such as monensin and ouabain. A variety of known methods can be employed for conjugating the cytotoxic agent to the carbohydrate-binding agent.

The composition of the invention comprises preferably one of said agent or agents disclosed above. However, the agent and agents are not limited to only those agent or agents. Said agent or agents might also be surfactants, for example sodium dodecylsulfate (SDS) and polyoxyethylene sorbitan fatty acid esters (Tween), and hypotonic solutions, for example pure water. Surfactants and hypotonic solutions destroy lens epithelial cells by rupturing the cell membrane wall. Chemical and enzymatic agents that release lens epithelial cells from the capsular bag membrane are also suitable for use in the present invention. Such agents include ethylene diamine tetraacetic acid (EDTA), trypsin, disintegrins, arginineglycine-asparagine (RGID) peptide analogs as well as antibodies directed against cell attachment receptors.

The present invention relates also to a method of introducing one or more agents into an eye during a process of freeing the lens from the capsular bag in order to treat the capsular bag or cells thereupon for the purpose of preventing residual lens epithelial cell proliferation and capsular opacification (CO) that might follow extracapsular extraction of a cataractous lens and due to fact that the lens is left in the capsular bag, the localization of said one or more agents in the capsular bag is improved. Thus, said one or more agents will be able to perform their inhibiting action in the space created between the capsule and the natural lens where most if not all of the lens epithelial cells, which will remain after the lens extraction are located.

Said method comprises the steps of creating an incision within an eye and a capsulorhexis in a capsular bag of said eye; inserting at least a portion of cannula through said capsulorhexis to perform the separation process i.e. the lens is separated from the capsular bag. Sealing means for the capsulorhexis can optionally be employed to reduce the risks of inadvertent distribution of toxic agents to the tissues of the anterior chamber. Suitable such means to introduce with the inventive method are found in WO 02/43632, PCT/EP01/13746 and WO 00/49976, which are all incorporates by reference. The sterile ophthalmic composition comprising one or more agents capable of inhibiting lens epithelial proliferation dissolved in a physiologically isotonic solution composition is injected whereby the lens is separated form the capsular bag and whereby said agent or agents will be able to perform its/their cell proliferation inhibiting action in the region adjacent to the capsular bag where cell proliferation is to be expected. The said agent or agents will destroy the lens epithelial cells and may also affect other cells within the eye if they leach from the capsular bag. However, since said agent or agents are introduced into the capsular bag during the process in which the lens is separated from the capsular bag by using hydrodissection or similar techniques the risk of leakage of said agent or agents into the eye is minimized due to compartmentalization in the space between the capsule and the lens. Furthermore, due to the fact that there is reduced risk of leakage of any appreciable amount of said agent or agents, said agent or agents can be left in the capsular bag during the time they need to perform their cell proliferation inhibiting action. The length of time required for the said agent or agents to inhibit lens epithelial cell proliferation and migration depends on a number of factors, including, but not limited to, the concentration of the agent or agents in the composition, the agent or agents selected and the toxicity of the agent or agents. Thus, the length of time can be from seconds to minutes, preferably less than 10 minutes, more preferably less than 3 minutes, most preferably less than 30 seconds. The agents can be introduced, as earlier mentioned, through a standard cannula or any type of phacoemulsification or irrigation/aspiration instrument that has exchangeable surgical probes or a purpose built hydrodissection instrument.

The most preferred method to separate the natural crystalline lens from the capsular bag is, as mentioned earlier, hydrodissection. The hydrodissection technique most preferably used in the invention is a cortical cleaving hydrodissection technique. This technique is e.g. described in Fine's article from 1992 (“J. Cataract Refract Surgery”; vol. 18; September; p 508-512) incorporated herein in its entirely by reference. In this technique a fluid wave is injected just under the anterior capsule in such a way that it separates the lens from the capsular bag. The cortical cleaving technique is also disclosed in Apple's article in “Survey of Ophthalm.” (37 (2); 1992; p. 73-116) and in Faust's article in “Am. Intraocular. Implant Soc. J.” (vol. 10; winter; 1984; p. 75-77). All of these articles are incorporated herein in their entirely by references. Other hydrodissection techniques are of course also possible for performing this invention for example the multilamellar hydrodissection technique disclosed in “J. Cataract Refract. Surgery” (Koch et al.; vol 16; September; 1990; p. 559-562) and the selective hydrodissection technique disclosed in “Ophthalmic. Surgery” (Blumenthal et al.; 1992; 23(10), p. 699-701) and the hydrodissection technique described in “J. Cataract Surg.” (Blumenthal et al., vol. 17; March 1991; p. 211-217). Thus, a person skilled in the art will easily realize that other hydrodissection techniques are possible.

The natural crystalline lens and the composition are then removed after a certain time either together or separately. When the lens has been separated from the capsular bag and the composition has been removed, the lens is fragmented by using e.g. an ultra sonic probe or an impeller probe equipped with a high-speed impeller interfaced with irrigation and aspiration capabilities as described in U.S. Pat. Nos. 5,437,678 and 5,690,641, each incorporated herein in its entirety by reference. However, alternatively any other surgical lens removal instrument may be used. The implant is inserted into the capsular bag. The implant is preferably either a conventional intraocular lens (IOL) or an injectable ophthalmic material that is optionally crosslinked and thus forms a lens inside the capsular bag.

The implant disclosed in the present is an artificial lens, which can be a foldable lens or non-foldable lens made from polymethylmethacrylate, homo or co-polymers of acrylates or methacrylates or other substituted acrylates, whether hydrophobic or hydrophilic or polysiloxane polymers. The lens can also be made from an injectable ophthalmically material that can undergo crosslinking to a final lens implant following its injection into the capsular bag, such as material is disclosed in, but not limited to, WO 99/47185, WO 00/22459, WO 00/22460, WO 01/77197, WO 01/76651 and PCT/EP02/07875 that are hereby incorporated as references.

The methods of the present invention for the prevention of CO are described in still greater detail in the Example that follows. The example is not supposed to be limited to the invention. The balanced salt solution used in these experiments was Alcon BSS®. It comprises sodium chloride (NaCl), potassium chloride (KCl), calcium chloride (CaCl₂.H₂O), magnesium chloride (MgCl₂.6H₂O), sodium acetate (C₂H₃NaO₂.3H₂O), and sodium citrate dihydrate (C₆H₅Na₃O₇.2H₂O). Each milliliter contains: sodium chloride 0.64%, potassium chloride 0.075%, calcium chloride 0.048%, magnesium chloride 0.03%, sodium acetate 0.39%, sodium citrate 0.17%, sodium hydroxide and/or hydrochloric acid (to adjust pH), and water.

### EXAMPLE

This example serves to visualize the method of the present invention. Therefore in lieu of a toxic agent, tryptan blue dye is added to a BSS® solution and the composition is thoroughly mixed.

An eye is placed under an operating microscope.

A clear cornea incision is performed and a capsulorhexis opening is made in the capsule. A paracentesis at the limbus is then made. A 27-gauge cannula is attached to a 10 ml syringe comprising the composition. The cannula is inserted through the paracentesis and subsequently through the capsulorhexis between the capsular bag and the lens cortex. The plunger of the syringe is depressed to perform hydrodissection and thereby also inject the composition between the capsular bag and the lens cortex and thus separating the lens from the capsular bag. It can be verified by visual inspection through the operating microscope that the composition is spread out in the space created between the capsular bag and the lens, since the composition is colored. The composition is then removed from the capsular bag together with the lens by the means of phacoemulsion. An artificial lens is then injected.

1-32. (canceled)

33. A sterile ophthalmic composition comprising one or more agents capable of inhibiting lens epithelial proliferation, dissolved in a physiologically isotonic solution.

34. A composition according to claim 33, wherein the physiological isotonic solution comprises a balanced salt solution.

35. A composition according to claim 33, wherein the one or more agents include one or more cytotoxic agents.

36. A composition according to claim 35, wherein the one or more cytotoxic agents are selected from the group consisting of saporin, ricin, methotrexate, 5-fluorouracil, daunomycin, doxorubicin, mitoxanthrone, vinca alkaloids, vinblastine, colchicine, cytochasins, monensin, mitomycin and ouabain.

37. A composition according to claim 36, wherein the one or more cytotoxic agents include 5-fluorouracil.

38. A composition according to claim 36, wherein the one or more cytotoxic agents include saporin.

39. A composition according to claim 36, wherein the one or more cytotoxic agents include mitomycin.

40. A composition according to claim 36, wherein the one or more cytotoxic agents include doxorubicin.

41. A composition according to claim 33, wherein the one or more agents comprise a molecule of nucleic acid comprising a gene coding for an agent inducing the death of the lens epithelial cells, under a transcriptional control specific to said cells.

42. A composition according to claim 41, wherein the gene coding for an agent inducing the death of the lens epithelial cells is selected from the group consisting of genes coding for a protein inducing cell death by necrosis and genes coding for proteins toxic for the lens epithelial cells.

43. A composition according to claim 42, wherein the gene coding for an agent inducing the death of the lens epithelial cells is a gene coding for an agent inducing apoptosis, or a gene involved in the process of apoptosis.

44. A composition according to claim 41, wherein the gene coding for an agent inducing the death of the lens epithelial cells is selected from the group consisting of genes coding for p53, BAX, FLICE (caspase 8), TRAIL and TRAIL-R.

45. A composition according to claim 34, wherein the one or more agents include a molecule of nucleic acid comprising a gene coding for an agent inducing the death of the lens epithelial cells, under a transcriptional control specific to said cells.

46. A composition according to claim 33, wherein the one or more agents comprise a vector comprising a molecule of a nucleic acid selected from the group consisting of genes coding for a protein inducing cell death by necrosis and genes coding for proteins toxic for the lens epithelial cells.

47. A composition according to claim 46, wherein the vector is an adenovirus vector.

48. A composition according to claim 33, wherein the one or more agents include one or more basement membrane binding agents conjugated to one or more cytotoxic agents.

49. A composition according to claim 48, wherein the one or more agents includes one or more basement membrane binding agents conjugated to one or more cytotoxic agents selected from the group consisting of ribosomal inhibitory proteins, antimitotic drugs and ionophores.

50. A composition according to claim 49, wherein the one or more agents includes one or more basement membrane binding agents selected from the group consisting of poly-L-lysine, poly-D-lysine, fibronectin, laminin, type I, II, III or IV collagen, thrombospondin, vitronectin, polyarginine and platelet factor IV, conjugated to one or more cytotoxic agents.

51. A composition according to claim 50, wherein the one or more agents includes poly-L-lysine or poly-D-lysine as a basement membrane binding agent conjugated to one or more cytotoxic agents.

52. A composition according to claim 48, wherein the one or more agents includes one or more cytotoxic agents selected from the group consisting of ribosomal inhibitory proteins, antimitotic drugs and ionophores.

53. A composition according to claim 49, wherein the one or more agents include one or more ribosomal inhibitory proteins as cytotoxic agents.

54. A composition according to claim 33, wherein the one or more agents comprise surfactants.

55. A composition according to claim 33, wherein the one or more agents comprise divalent cation chelators.

56. A composition according to claim 33, wherein the one or more agents comprise analogs or antibodies directed against cell attachment receptors.

57. A method of preventing capsular opacification comprising:
a) creating an incision within an eye and a capsulorhexis in a capsular bag of the eye;
b) inserting at least a portion of a cannula in the capsulorhexis;
c) separating the natural crystalline lens from the capsular bag by injecting through the cannula a sterile ophthalmic composition in a manner that generates a space between the capsular bag and the natural crystalline lens wherein the composition comprises one or more agents capable of inhibiting lens epithelial proliferation dissolved in a physiologically isotonic solution;
d) retaining the composition in the space between the capsule and the natural crystalline lens, for a sufficient time for said composition to effectively kill or render non-proliferative lens epithelial cells in or adjacent to the space;
e) removing the lens and the composition; and
f) inserting an implant into the capsular bag.

58. A method according to claim 57 wherein the one or more agents include one or more cytotoxic agents.

59. A method according to claim 57, wherein the one or more agents comprise a molecule of nucleic acid comprising a gene coding for an agent inducing the death of the lens epithelial cells, under a transcriptional control specific to said cells.

60. A method according to claim 57, wherein the one or more agents comprise one or more basement membrane binding agents conjugated to one or more cytotoxic agents.

61. A method according to claim 57, wherein the one or more agents comprise surfactants.

62. A method according to claim 57, wherein the one or more agents comprise divalent cation chelators.

63. A method according to claim 57, wherein the one or more agents comprise analogs or antibodies directed against cell attachment receptors.

64. A method according to claim 57, wherein the implant is an artificial lens.

65. A method according to claim 64, wherein the lens is a foldable lens or non-foldable lens made from a material comprising polymethylmethacrylate, homo or copolymer of acrylate, methacrylate or other substituted acrylate, or polysiloxane.

66. A method according to claim 57, wherein the implant comprises an injectable ophthalmically-acceptable material that can undergo crosslinking to a final lens implant following its injection into the capsular bag.

67. A method according to claim 57, wherein the lens is separated from the capsular bag by hydrodissection.

68. A method of preventing capsular opacification, comprising administering to a capsular bag a sterile ophthalmic composition comprising one or more agents capable of inhibiting lens epithelial proliferation dissolved in a physiologically isotonic solution.

69. A method according to claim 68 wherein the one or more agents include one or more cytotoxic agents.

70. A method according to claim 68, wherein the one or more agents comprise a molecule of nucleic acid comprising a gene coding for an agent inducing the death of the lens epithelial cells, under a transcriptional control specific to said cells.

71. A method according to claim 68, wherein the one or more agents comprise one or more basement membrane binding agents conjugated to one or more cytotoxic agents.

72. A method according to claim 68, wherein the one or more agents comprise surfactants.

73. A method according to claim 68, wherein the one or more agents comprise divalent cation chelators.

74. A method according to claim 68, wherein the one or more agents comprise analogs or antibodies directed against cell attachment receptors.
